(19) United States
(12) Patent Application Publication
(10) Pub. No.: US 2008/0038284 A1
(43) Pub. Date: Feb. 14, 2008

(54) Human Immunodeficiency Virus Vaccine

(76) Inventors: Barton F. Haynes, Durham, NC (US); Hua-Xin Liao, Chapel Hill, NC (US)

Correspondence Address: NIXON & VANDERHYE, PC, 901 NORTH GLEBE ROAD, 11TH FLOOR, ARLINGTON, VA, 22203, US

(21) Appl. No.: 11/666,732

(22) PCT Filed: Nov. 7, 2005
(86) PCT No.: PCT/US05/40160

§ 371 Date: May 2, 2007

Related U.S. Application Data
(60) Provisional application No. 60/625,720, filed on Nov. 8, 2004.

(51) Int. Cl. A61K 39/12; A61K 31/7052; A61P 37/00; C07K 2/00; C12N 15/63; C07H 21/00; A61K 38/02

(52) U.S. Cl. 424/184.1; 435/320.1; 514/2; 514/44; 530/300; 536/23.5

(57) ABSTRACT

The present invention relates, in general, to human immunodeficiency virus (HIV) and, in particular, to an HLA-based HIV vaccine.

This application claims priority from U.S. Provisional Application No. 60/625,720 filed Nov. 8, 2004, the entire content of which is incorporated herein by reference.

### TECHNICAL FIELD

The present invention relates, in general, to human immunodeficiency virus (HIV) and, in particular, to an HLA-based Th-CTL vaccine.

### BACKGROUND

As the HIV epidemic continues to spread world-wide, the need for an effective HIV vaccine remains urgent. The extraordinary ability of HIV to mutate, the inability of many currently known specificities of anti-HIV antibodies to consistently neutralize HIV primary isolates, and the lack of a complete understanding of the correlates of protective immunity to HIV infection have impeded efforts to develop an HIV vaccine having the desired effectiveness.

Although a majority of HIV-infected subjects develop acquired immunodeficiency syndrome (AIDS), approximately 10-15% of patients are AIDS-free after 10 years of infection, and are termed non-progressors to AIDS (Sheppard et al, AIDS 7:1159-66 (1993), Phair, AIDS Res. Human Retroviruses 10:883-885 (1994)). Of those that do develop AIDS, approximately 10% of HIV-infected patients progress to AIDS within the first two to three years of HIV infection, and are termed rapid progressors to AIDS (Sheppard et al, AIDS 7:1159-66 (1993), Phair, AIDS Res. Human Retroviruses 10:883-885 (1994)). The initial characterization of anti-HIV immune responses in non-progressors and rapid progressors to AIDS has provided some insight into what may be the correlates of protective immunity to HIV.

In general, rapid progressors to AIDS have lower levels of antibodies to HIV proteins (Sheppard et al, AIDS 7:1159-66 (1993), Pantaleo et al, N. Engl. J. Med. 332:209-216 (1995), Cao et al, N. Eng. J. Med. 332:201-208 (1995)), and low or absent antibodies that neutralize autologous HIV isolates (Pantaleo et al, N. Engl. J. Med. 332:209-216 (1995), Cao et al, N. Eng. J. Med. 332:201-208 (1995)). Anti-HIV CD8+ CTL activity is present in peripheral blood T cells of rapid progressors, although one study has found low levels of memory CD8+ CTL by precursor frequency analysis in rapid progressors versus non-progressors (Pantaleo et al, Nature 370:463-467 (1994), Rinaldo, personal communication (1995)). Plasma levels of HIV virions are generally higher in rapid progressors compared to non-progressors, and rapidly replicating HIV strains are isolated more frequently from rapid progressors (Lee et al, J. AIDS 7:381-388 (1994), Mellors et al, Ann. Intern. Med. 122:573-579 (1995), Jurriaans et al, Virology 204:223-233 (1994)), either as a consequence of immunodeficiency and selection of more virulent HIV variants, or as a consequence of more virulent HIV variants infecting rapid progressors (Sullivan et al, J. Virol. 69:4413-4422 (1995)). Taken together with data that the fall in plasma viremia in primary HIV infection correlates with the presence of CD8+ anti-HIV CTL activity (Borrow et al, J. Virol. 68:6103 (1994)), these data suggest that anti-HIV CD8+ CTL that kill HIV-infected cells and antibodies that broadly neutralize HIV primary isolates, might be protective anti-HIV immune responses in uninfected individuals subsequently exposed to HIV (Haynes et al, Science 271:324-328 (1996), Haynes, Science 260:1279-1286 (1993)).

It has been suggested that less effective anti-HIV CD8+ CTL responses may be oligoclonal regarding TCR Vβ usage and targeted at several non-immunodominant HIV CTL epitopes, whereas more effective anti-HIV CTL responses may be polyclonal and targeted at fewer immunodominant epitopes (Rowland-Jones et al, Nature Medicine 1:59-64 (1995), Nowak et al, Nature 375:606-611 (1995)). Taken together with data that suggest the inheritance of certain HLA-encoded or other host genes may be associated with either rapid progression or non-progression to AIDS (Haynes et al, Science 271:324-328 (1996)), these data suggest that host gene expression may determine the quality and/or quantity of host anti-HIV immune responses.

Potent non-HLA restricted CD8+ T cell anti-HIV activity that suppresses the ability of HIV to replicate has been described by Levy et al (Walker et al, Science 234:1563-1566 (1986)). This CD8+ “HIV suppressor” activity is initially present in rapid progressors, then declines with the onset of AIDS (Walker et al, Science 234:1563-1566 (1986)), and may be mediated in part by cytokines such as IL-16 (Baier et al, Nature 378:563 (1995)), and by the chemokines, RANTES, MIP-1a and MIP-1b (Cocchi et al, Science 270:1811-1815 (1995)). Berger and colleagues have recently discovered a novel host molecule termed fusin, that is required for T cell tropic HIV to infect CD4+ T cells, and has significant homology with a known chemokine receptor, the IL8 receptor (Feng et al, Science 272:872-877 (1996)).

Thus, for induction of CD8+ “HIV suppressor” cells, CD8+ CTL and CD4+ T helper cells by an HIV immunogen, what is most likely needed are immunogens that induce these anti-HIV responses to a sufficient number of HIV variants such that a majority of HIV variants in a geographic area will be recognized.

A key obstacle to HIV vaccine development is the extraordinary variability of HIV and the rapidity and extent of HIV mutation (Win-Hobson in The Evolutionary biology of Retroviruses, SSB Morse Ed. Raven Press, NY, pgs 185-209 (1994)). Recent data in patients treated with anti-retroviral drugs have demonstrated that HIV variants emerge rapidly after initiation of treatment and can be isolated from peripheral blood as early as 3 weeks after initiation of drug treatment (Wei et al, Nature 373:117-122 (1995), Ho et al, Nature 373:123 (1995)). Moreover, up to 10⁹ new HIV virions are produced in an infected individual per day, and the half-life of HIV cruasispecies is approximately 2 days (Wei et al, Nature 373:117-122 (1995), Ho et al, Nature 373:123 (1995)).

Myers, Korber and colleagues have analyzed HIV sequences worldwide and divided HIV isolates into groups or clades, and provided a basis for evaluating the evolutionary relationship of individual HIV isolates to each other (Myers et al (Eds), Human Retroviruses and AIDS (1995), Published by Theoretical Biology and Biophysics Group, T-10, Mail Stop K710, Los Alamos National Laboratory, Los Alamos, N. Mex. 87545). The degree of variation in HIV protein regions that contain CTL and T helper epitopes has also recently been analyzed by Korber et al, and sequence variation documented in many CTL and T helper epitopes among HIV isolates (Korber et al (Eds), HIV Molecular Immunology Database (1995), Published by Theoretical Biology and Biophysics Group, Los Alamos National Laboratory, Los Alamos, N. Mex. 87545). (See also Korber et al (Eds), HIV Molecular Immunology Database (1999-2005), Published by Theoretical Biology and Biophysics, Group T-10, Los Alamos National Laboratory, Los Alamos, N. Mex., Vastutomi et al, J. Virol. 69:2279 (1995)).

A new level of HIV variation complexity was recently reported by Hahn et al. by demonstrating the frequent recombination of HIV among clades (Robinson et al, J. Mol. Evol. 40:245-259 (1995)). These authors suggest that as many as 10% of HIV isolates are mosaics of recombination, suggesting that vaccines based on only one HIV clade will not protect immunized subjects from mosaic HIV isolates (Robinson et al, J. Mol. Evol. 40:245-259 (1995)).

The large number of HIV variants available for transmission and the possible immunodominant nature of what may be protective anti-HIV T cell responses has suggested the need for consideration of development of HLA-based HIV subunit vaccines (Palker et al, J. Immunol. 142:3612-3619 (1989), Berzofsky, FASEB Journal 5:2412 (1991), Haynes et al, Trans. Assoc. Amer. Phys. 106:33-41 (1993), Haynes et al, AIDS Res. Human. Retroviral. 11:211 (1995), Ward et. al, Analysis of HLA Frequencies in Population Cohorts for Design of HLA-Based HIV Vaccine, IV-10-IV-16, HIV Molecular Immunology Database (1995), Korber et al (Eds), Theoretical Biology and Biophysics, Group T-10, Mail Strop K710, Los Alamos National Laboratory, Los Alamos, N. Mex., Cease et al, Ann. Rev. Immunol. 12:923-989 (1994)). The present invention provides such a vaccine.

### SUMMARY OF THE INVENTION

The present invention relates to an HLA-based Th-CTL vaccine against HIV. The invention also relates to a method of immunizing a patient against HIV using the HLA-based Th-CTL vaccine.

Objects and advantages of the present invention will be clear from the description that follows.

### BRIEF DESCRIPTION OF THE DRAWINGS

FIGS. 1A-1D. C4-V3 Th-CTL Peptides Induce HLA B7 Reactive CD8+ CTL in Normal HIV-1 Seronegative Humans. FIGS. 1A and 1C show specific lysis from in vivo immunization and in vitro restimulation against each of the V3 B7 CTL epitope variants. BLCL=B lymphoblastoid cell (BCLC) no peptide coating control. C4=C4 Th determinant peptide on BCLC, V3MN, V3RF, V3EV91, and V3Can0A are the B7 CTL epitope variant peptide coated on BCLC. Data show patient in FIG. 1A responded to 1 of 4 B7 CTL epitope variants (the HTV EV91 variant) while the patient in FIG. 1C responded to 3 of 4 B7 epitope variants (HIV MN, EV91 and Can0A). FIGS. 1B and 1D show 2 HLA B7 negative individuals that made no CTL response to the B7-restricted CTL peptide immunogen after both in in vivo immunization and in vitro restimulation.

### DETAILED DESCRIPTION OF THE INVENTION

The present invention relates to an HLA-based Th-CTL HIV vaccine. The invention further relates to a method of immunizing a patient against HIV by using such a vaccine.

The HLA-based vaccines of the invention can be designed based on available HLA databases. Results obtained in International Histocompatibility Testing Workshops, such as the most recent ones (Histocompatibility Testing 1980, Teresaki (Ed.), UCLA Tissue Typing Laboratory, Los Angeles, Calif. (1980), Histocompatibility Testing 1984, Albert et al (Eds.), Springer-Verlag, Berlin (1984), Immunobiology of HLA, 2 volumes, Dupont (Ed.), Springer-Verlag, New York, (1989), HLA 1991, 2 volumes, Tsuji et al (Eds.), Oxford University Press, Oxford (1992), Asian Pac. J. Allergy Immunol. 22:143-151 2004), J. Med. Assoc. Thai. 86:S230, S236 (2003)), provide such a database.

The International Histocompatibility Workshop data (such as Histocompatibility Testing 1984, Albert et al (Eds.), Springer-Verlag, Berlin (1984), HLA 1991, 2 volumes, Tsuji et al (Eds.), Oxford University Press, Oxford (1992)), supplemented with published data from selected laboratories (such as Williams et al,. Human Immunol. 33:39-46 (1992), Chandanayingyong et al, In Proceedings of the Second Asia and Oceania Histocompatibility Workshop Conference, Simons et al (Eds.), immunopublishing, Toorak, pgs. 276-287 (1983)) provide an estimate of the frequencies of HLA alleles that have been shown to serve as restriction elements for HIV CTL epitopes (HIV Molecular Immunology Database (1995), Korber et al (Eds.), Los Alamos National Laboratory: Published by Theoretical Biology and Biophysics Group, Los Alamos National Laboratory, Los Alamos, N. Mex. 87545.). (See also Korber et al (Eds), HIV Molecular Immunology Database (1999-2005), Published by Theoretical Biology and Biophysics, Group T-10, Los Alamos National Laboratory, Los Alamos, N. Mex., Vastutomi et al, J. Virol. 69:2279 (1995)). Table 1 summarizes these frequencies for the four populations: African Americans, North American Indians, USA Caucasians, and Thais, used here for purposes of exemplification. Section II of the Los Alamos HIV epitope database of Korber et al (HIV Molecular Immunology Database (1995), Los Alamos National Laboratory: Published by Theoretical Biology and Biophysics Group, Los Alamos National Laboratory, Los Alamos, N. Mex. 87545) lists the CTL epitopes by HLA restriction element. Using these two sets of data and the Hardy-Weinberg theorem (Hardy, Science 28:49-50 (1908)), the proportion of each of the four populations that would be predicted to present peptides to the immune system if a limited number of HIV epitopes were included in a vaccine designed specifically for that population can be estimated. A similar calculation for a vaccine designed to be immunogenic for all four populations has been made. These results are presented in Table 2.

The strategy that can be used in this analysis is to first identify the most frequent restriction elements in the population under consideration for vaccination (or common to the 4 populations), to identify peptides that are presented by more than one HLA allele, and then to seek commonality between these two lists. Probability calculations then utilize the frequencies of the commonality alleles supplemented by those of additional high frequency alleles in the population. Alleles can be added until the proportion of the individuals in the population carrying one or more of the alleles in the list is at an acceptable level, for instance, greater than 90% in the examples. The aim is to maximize the sum of the HLA gene frequencies that recognize the least number of different HIV peptides to be included in an HIV immunogen. The next step is to choose the peptides associated with the restricting allele. In some instances,only one peptide is associated with an allele while in others, multiple peptides are presented by the same allele.

Criteria that can be used choosing which immunogenic epitopes to be included in a preventive HIV immunogen are listed below:

1. Peptides reported to be immunogenic in situations thought to reflect protection from retroviral infection or protection from retroviral-induced immunodeficiency disease (i.e., in non-progressors to AIDS).

2. Peptides presented to the immune system by HLA restricting elements reported to be associated with non progression to AIDS (for example, Haynes et al, Science 171:324-328 (1996)).

3. Peptides reported to be “immunodominant” stimulators of HLA class I-restricted anti-HIV CTL responses (Nowak et al, Nature 375:606-611 (1995)).

4. Peptides reported presented by several disparate HLA class I allotypes.

For the four population cohorts considered in detail here by way of example, as few as 2 and as many as 5 epitopes are required to achieve a theoretical protection level of at least 90% (Table 2). The different numbers of required epitopes reflect the relative amounts of HLA Class I polymorphism observed in the different ethnic groups and presentation of a peptide by multiple HLA class I molecules. To date, HIV peptides have been associated only with HLA restriction elements that are infrequent in some populations. As more data are accumulated for other epitopes, some that are associated with higher frequency restriction elements may be identified.

A comparison between the individual and combined populations (Table 2) demonstrates that relatively little is gained by including epitopes that are associated with low frequency alleles. The proportion of individuals protected approaches 100% asymptotically so that even adding on epitopes associated with high frequency alleles adds little to the proportion as this level is approached. This is illustrated by the North American Indians where including 6 more epitopes associated with 5 very low frequency alleles and one intermediate frequency allele in the combined theoretical vaccine adds only 3.0% protection.

U.S. Pat. No. 5,993,819 (the contents of which is incorporated herein by reference) also includes a description of the steps involved in the development of an HLA-based HIV vaccine. In Table XXVI of that patent, the following vaccine formula is provided which is equally applicable here:
Th₁-X₁, Th₂-X₂, Th₃-X₃, . . . Th_{N}-X_{N}
where Th=immunodominant T helper epitopes and X=MHC Class I CTL epitopes. In the context of a preferred embodiment of the invention, Table 3 provides specific TH-X peptides (see vaccines 6, 8 and 10, particularly vaccines 6 and 8) that can be admixed, formulated with a pharmaceutically acceptable carrier, and adjuvant, as appropriate, and administered to a patient in order to effect immunization. The optimum amount of each peptide to be included in the vaccine and the optimum dosing regimen can be determined by one skilled in the art without undue experimentation.

As an alternative to using mixtures of individual Th-X peptides, the vaccine of the presently preferred embodiment can also take the form of a linear array of Th-X epitopes (see the linear arrays of MVA 6-10 in Table 4, particularly MVA 6 and MVA 8), preferably, expressed in a modified Vaccinia ankara (Zentralbl. Bakterial 167:375-390 (1978); Nature Med. 4:397-402 (1988)) or other live vector such as an adenoviral vector or a canary pox vector (Weinhold et al, Proc. Natl. Acad. Sci. 94:1396-1401 (1997)). Upon expression with HIV gag p55, pseudovirons (particles) are produced (see, for example, the linear arrays of MVA 7 and 9 in Table 4). Standard procedures can be used to formulate the vaccine (e.g., with a carrier and, as appropriate, with an adjuvant) and optimum dosing regimes can be determined by one skilled in the art without undue experimentation.

In a further embodiment, the vaccine of the present invention includes MHC Class I restricted cytotoxic T lymphocytes (CTL) epitopes from HIV p17 and p24 gag regions. Known HIV CTL epitopes and their MHC restricting elements are listed in “HIV Molecular Immunology Database, 1999” (Korber, BTM, Brander, C., Haynes, B. F. et al Editors, Published by the Theoretical Biology and Biophysics Group T-10, Mail Stop K710 Los Alamos National Laboratory, Los Alamos, N. Mex. 87545). The CTL regions designated CTL-J, CTL-K, CTL-L and CTL-M are selected for Vaccine 11 in Table 3. The full peptide has been designed to have at the N-terminus of the epitope the optimal Th determinant, ThA E9V from HIV gp120 C4 region. The restricting elements predicted to respond to these peptides are listed to the right in Table 3. Thus, a practical HIV gag CTL immunogen is set forth in Table 6, with A-Th/A-CTL and B-Th/B-CTL peptides mixed with the peptides in Vaccine 11. The 25 HLA Class I molecules predicted to recognize the peptides in the mixture of peptides in Table 6 are listed at the bottom of the table.

In a further embodiment, the immunogenic composition of the invention includes one or more of the peptides set forth in Tables 7 and 8, alone or in combination with one or more of the other peptides disclosed herein. For example, A*Th/M1-CTL, A*Th/M2-CTL, B-Th/L-CTL (referred to as B-Th/L2-CTL in Table 7) and B-Th/R-CTL, of subtype B consensus sequences, can be used with subtype B peptides ATh/A-CTL, B-Th/B-CTL, CTh/C-CTL, and A*Th/J-CTL, for an eight-valent immunogen. A-n eight-valent immunogen can also comprise, for example, C-Th/C-CTL from Table 3, A-Th/A-CTL, B-Th/B-CTL, and A*Th/J-CTL from Table 6 and A*Th/L2-CTL, A*Th/M1-CTL, A*Th/M2-CTL, and A*Th/R-CTL from Table 8.

For peptides in Table 7, subtype B consensus sequences in the Th-CTL immunogen format were chosen that include multiple CTL epitopes recognized by the most common HLA restricting elements. In this regard, 98% of African-Americans and 99% of US caucasian can be predicted to recognize at least one of the epitopes in Table 7. Also selected were CTL epitopes from different HIV-1 proteins to expand the regions in the HIV-1 genome recognized. Thus, six “hot spots” were selected for CTL epitopes from HIV-1 Gag, Env and Pol proteins (Table 7). To enhance the immunogenicity of these CTL peptides and to provide T cell help, the C4 Env Th epitope (A*Th, see Table 7) or the GTH1 Gag Th epitope (BTh, see Table 7) can be used as these epitopes have been documented for their ability to induce Th responses in multiple species and, for the C4 peptide, in outbred humans (Palker et al, J. Immunol. 142:3612-3619 (1989), Weaver et al, AIDS Vaccine, Abstr. 43, p. 57, New York Sep. 18-21 (2003), Korber et al, AIDS. Res. and Hum. Retrovir., 8:1461-1465, (1992)).

Complex immunogens made up of CTL sequences, for example, from the Los Alamos Database (Korber, BTM, Brander, C., Haynes, B. F. et al Editors, Published by the Theoretical Biology and Biophysics Group T-10, Mail Stop K710 Los Alamos National Laboratory, Los Alamos, N. Mex. 87545) can be prepared by adding to the sequences in Table 6, new sequences from CTL epitopes in envelop, rev, nef, tat, pol and other regions of the HIV genome. These sequences can be formulated with T helper sequences as above in Table 6 (generic Th-X1, Th-X2 . . . Th-Xn), or can be delivered in shorter sequences of X1, X2 . . . Xn, with T cell help being delivered by an appropriate adjuvant. In these generic designs, Th represents a helper T cell epitope, and X represents a HLA Class I restricted CTL epitope.

At each CTL sequence, there are many variants that can be included in the peptide mix in the above vaccine designs, in order to provide CTL that attack a sufficient number of HIV variants to prevent infection or to control infection. Variants are listed for each HIV Clade in the Los Alamos database for HIV sequences, “Human Retroviruses and AIDS”, Kuiken, C, Foley, B et al Editors, Published by the Theoretical Biology and Biophysics Group T-10, Mail Stop K710 Los Alamos National Laboratory, Los Alamos, N. Mex. 87545.

Since different geographic locations around the world have different HIV Clades infecting patient cohorts, the above peptide design can be modified to be appropriate for the Clade or Clades of HIV that are relevant for a particular geographic region. For example, the Los Alamos Database of HIV Sequences has a listing of sequences by country and by clade. Therefore, to design a CTL vaccine for Zambia in Sub-saharan Africa, the principles and general CTL epitope design described as above can be employed but using the most common or consensus sequences of the Clades and isolates in the data base from Zambia. This general strategy applyies to design of CTL immunogens for any geographic region of the world.

Peptides have the greatest use in focusing the immune response on many dominant and subdominant CTL epitopes of HIV, but may benefit from a prime from another type of immunogen. Thus, the sequences described above and given in Tables 3, 6, 7 and 8, as well as Zambian sequences and or sequences of epitopes from rev, nef, tat, pol or env, can also be constructed in linear arrays of CTL epitopes with or without T helper determinants, for example, in either plasmid DNA constructs or in live vector constructs such as Modified Vaccinia Ankara or in mycobacteria tuberculosis strains that are attenuated, such as BCG (Jacobs et al, Nature Medicine 2:334 (1996)). These DNA or live vectors with linear arrays of CTL epitopes can be used as either primes or boosts of peptides or of each other to optimally give CTL anti-HIV responses.

It will be appreciated that this embodiment of the invention includes not only the specific Th-X peptides, and derivatives thereof (e.g. as shown in MVA 7 and MVA 9 in Table 4), shown, for example, in Tables 3 and 4, but also includes variants of the indicated peptides as well, particularly variants of the CTL epitopes shown. The mixture or linear array of Th-X peptides can be used alone or as one component of a multi-component vaccine. It will also be appreciated that the peptides of the invention can be synthesized using standard techniques. It will also be appreciated that the vaccine of the present invention can take the form of a DNA vaccine the expression of which in vivo results in the expression of the peptides, or linear arrays of same, described above.

Suitable routes of administration of the present vaccine include systemic (e.g. intramuscular or subcutaneous). Alternative routes can be used when an immune response is sought in a mucosal immune system (e.g., intranasal). Appropriate routes and modes of administration can be selected depending, for example, on whether the vaccine is a peptide or DNA vaccine or combination thereof.

The peptides/polypeptides and nucleic acids of the invention can be present in a composition comprising, for example, a pharmaceutically acceptable carrier or diluent. The composition can also comprise, for example, an adjuvant and/or an immunomodulator (e.g., recombinant human granulocyte macrophage colony stimulating factor (GM-CSF)). The composition, which can be sterile, can be in dosage unit form.

A variety of adjuvants well known in the art can be used with the peptides/polypeptides and nucleic acids of the invention. Likewise, a variety of immunomodulators can be used. Adjuvants suitable for use with the peptides of the invention include, but are not limited to, oil-in-water emulsion-containing adjuvants, or water in oil adjuvants, such as mineral oil (IFA). Preferred oils include mineral oil and squalene. Suitable adjuvants can include CpG oligonucleotides and other agents (e.g., TRL 7, 8, and/or 9 agonists). (Tran et al, Clin. Immunol. 109:278-287(2003), US Appln Nos. 20030181406, 20040006242, 20040006032, 20040092472, 20040067905, 20040053880, 20040152649, 20040171086, 20040198680, 200500059619.)

Certain aspects of the present invention are described in greater detail in the Example that follows. (See also application Ser. No. 09/775,805 which is incorporated herein by reference.)

### EXAMPLE 1

Studies of Th-CTL Mutivalent in HLA B7+ Humans

Immunogenicity and Safety of the C4-V3 Th-CTL Polyvalent Immunogen in HIV Seropositive Patients with CD4+ T Cell Counts >500/mm3 (DATRI010). The DATRI010 human trial of the C4-V3 PV immunogen has been completed (Bartlett et al, AIDS Res. Hum. Retro. 12:1291-1300 (1998)). The immunogen was 4 Th-CTL peptides with the Th epitope the same in each peptide and the CTL peptide was four variants of a B7-restricted env CTL epi tope (Haynes, Res. Human Retro. 11:211-221 (1995), Beddows et al, J. Gen. Virol. 79:77-82 (1998), Table 5). Ten HIV-infected, HLA B7-positive patients with CD4+ T cells >500/mm3 were enrolled. Eight patients received 2 mg of C4-V3 polyvalent immunogen (i.e., 500 μg of each peptide) emulsified in incomplete Freund's adjuvant (Seppic ISA51) IM X5 over 24 weeks, and 2 controls received ISA51 IM alone. Vaccine recipients had excellent boosts of Th proliferative levels and neutralizing antibody levels to TCLA HIV (Bartlett et al, AIDS Res. Hum. Retro. 12:1291-1300 (1998)). However, in the setting of HIV infection, PBMC suspensions of immunized B7+ subjects had minimal direct CTL activity to the B7-restricted env CTL epitope in the immunogen to peptide coated targets or to vaccinia infected targets (i.e. the B7 gp120 CTL epitope was non-dominant in the setting of HIV infection) (Bartlett et al, AIDS Res. Hum. Retro. 12:1291-1300 (1998)).

AVEG020 Trial of Th-CTL C4-V3 Peptides in Seronegative Subjects. In conjunction with NIAID, DAIDS, DATRI and WLVP, AVEG020 “Phase 1 Safety and Immunogenicity Trial of C4-V3 Peptide Immunogen in HIV Seronegative Subjects” was carried out at Vanderbilt, Rochester, and Seattle as a multicenter trial (AVEG020 Doses: High Dose=4 mg total dose, 1 mg of each peptide per dose; Low Dose=1 mg total dose, 250 μg of each peptide per dose).

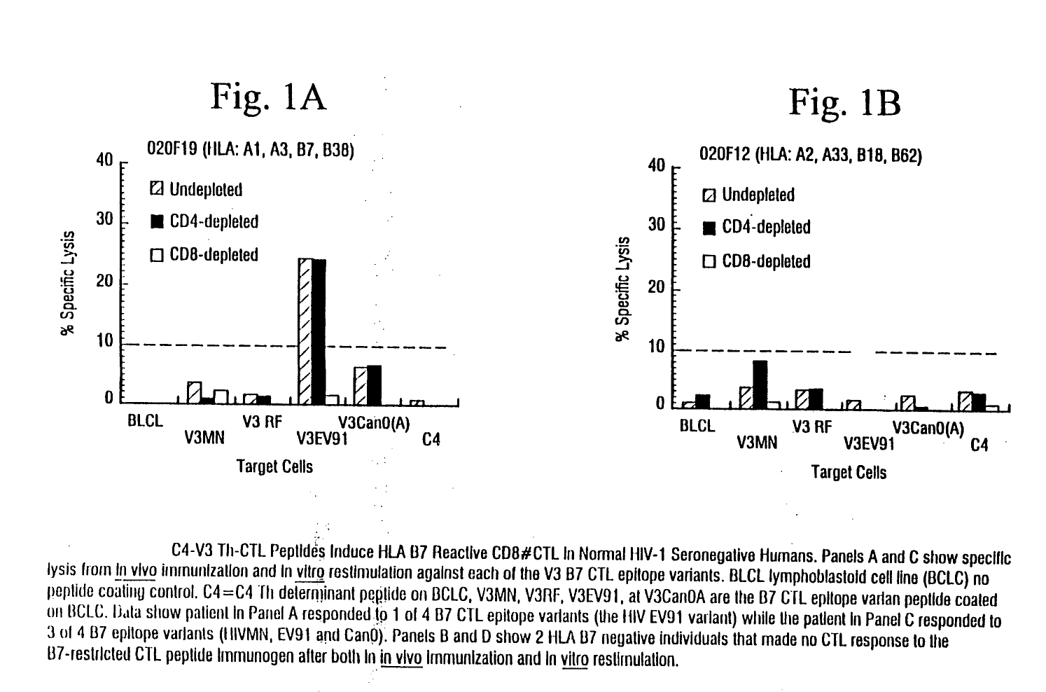

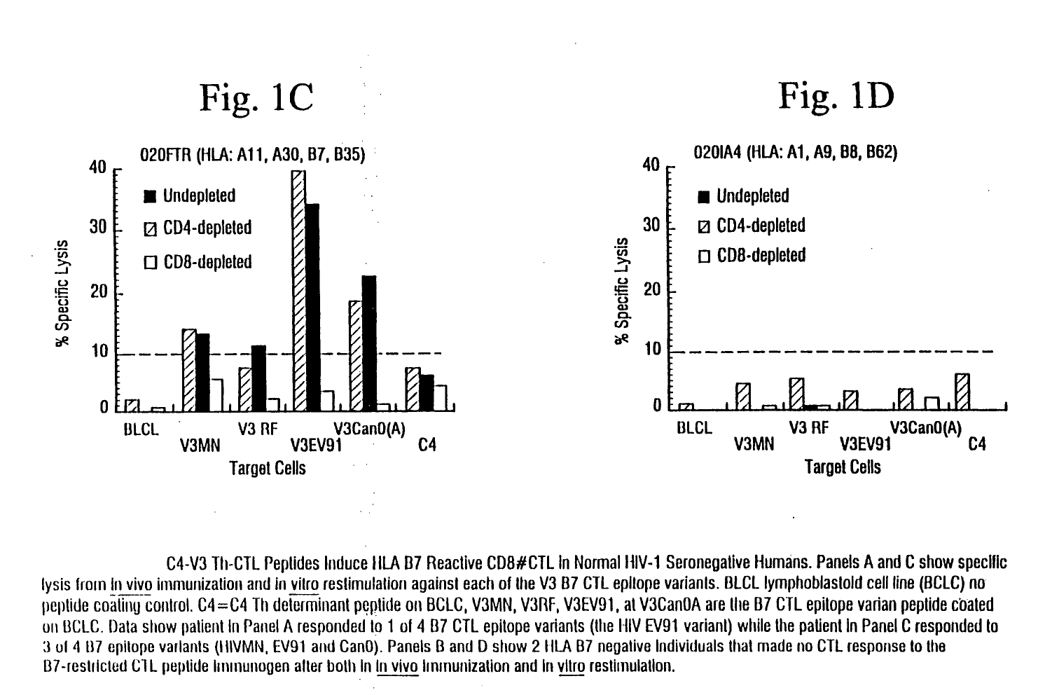

Studies were made of 13 subjects (9, B7− and 4 B7+) after two immunizations 250 μg of each peptide variant. Of 9 HLA B7-subjects, 0/9 had PB CTL activity to any of the peptide variants of the B7-restricted gp120 env CTL epitope in the immunogen (FIGS. 1B and 1D). In contrast, 2/4 HLA B7+ subjects had high levels of CTL activity to the B7 epitope that was mediated by CD8+ T cells and was MHC restricted after only two immunizations (FIGS. 1A and 1C). These data provided direct evidence that Th-CTL immunogens, when formulated in potent adjuvants, could induce MHC Class I-restricted CATL in humans. Whereas one subject responded to one of the 4 B7 epitope variants, the other subject (FIG. 1A) responded to 3 of the 4 CTL variants. These data demonstrated that a human host could respond to more than one CTL epitope variant in an immunogen, and indicated that epitope-based immunizations could be used to induce MHC Class I-restricted CD8+ CTL responses to CTL epitopes and to their variants.

### EXAMPLE 2

HIV Peptides

To enhance immunogenicity, the adjuvant, RC529-SE, and the immunomodulator, GM-CSF, are used. Individual materials are prepared to allow various mixtures to be administered.

Th/CTL Peptides

The CTL multi-epitope peptide (MEP) vaccine contains four peptides. Each peptide (27-47 amino acids) consists of one of four different regions from gag or nef that contain multiple overlapping CTL epitopes and one of four different HIV-derived T helper epitopes from env or gag. The design of this prototype vaccine includes epitopes bound by 15 different HLA types is projected to provide 85-95% coverage of the North American population depending on genetic background.

The peptide mixture is lyophilized. Prior to lyophilization, the four peptides are formulated in a solution of 3% mannitol and 12.5 mM succinic acid (pH 2).

Diluent

At the time of injection, the lyophilized peptides are reconstituted to the original volume with 12.5 mM sodium succinate (final pH, ˜4.8) and then mixed with other components.

Placebo

The placebo for the peptide vaccine is commercially available saline.

RC529-SE

RC-529 SE formulated at 500 μg/mL in 10% squalene (85.8 mg/mL), glycerol (22.7 mg/mL), D,L-alpha-tocopherol (0.5 mg/mL), egg phosphatidylcholine L-Lecithin egg (19.1 mg/mL), Poloxamer 188 [Pluronic F-68 Prill Surfactant] (0.9 mg/mL) and 0.025 M ammonium phosphate buffer (pH 5.1). The concentration of RC-529 must be appropriate to deliver a final dose of 50 μg and the concentration of the SE components must ensure a final squalene concentration of 1%.

GM-CSF

Recombinant human granulocyte macrophage colony stimulating factor, Leukine®, ready-to-use liquid formulation (Immunex), will be supplied as marketed.

Plasmids

HIV Gag Plasmid DNA (003/003M)

DNA (2 mg/mL) complexed with 0.25% bupivacaine in citrate buffer, pH 6.8. The plasmid encodes the HIV-1 strain HXB2 gag gene. In 003M, the change of a single nucleotide in the ori region of the original 003 backbone resulted in significant increase in manufacturing yields. This plasmid is hereinafter referred to as gag.

HuIL-12 Plasmid DNA (103/103M)

DNA (2 mg/mL) formulated as above. The dual promoter plasmid expresses both p35 and p40 chains of human interleukin 12 (HuIL-12). In 103M, the change of a single nucleotide in the ori region of the original 103 backbone resulted in significant increase in manufacturing yields. This plasmid is hereinafter referred to as HuIL-12.

HuIL-15 Plasmid (DNA 125M)

Purified DNA (2 mg/mL) complexed with 0.25% bupivacaine in citrate buffer, pH 6.5. The olasmid encodes the IL-15 sequence associated-with the long signal peptide (48 aa). 125M has been RNA optimized. The human leader sequence has been replaced by a rhesus leader sequence to maximize expression resulting in higher manufacturing yields. The plasmid is hereinafter referred to as HuIL-15.

Placebo

The placebo for DNA vaccine is commercially available saline.

Integration Analysis gag+HuIL-12 DNA

Results from the gag+HuIL-12 DNA biodistribution study indicated “high plasmid copy numbers” in tissue in several rabbits in the day 94 injection site sample. An integration study was performed on selected day 94 injection site tissue samples. The gag+HuIL-12 integration analysis was conducted on six selected test and two vehicle control tissue samples. The design of this study was to assess the potential integration of gag and HuIL-12 sequences into the genome in animal tissues following in vivo administration using quantitative Polymerase Chain Reaction (TaqMan® PCR) technique.

Preliminary results indicated that five of the six test samples were less than the lower limit of quantitation (LLOQ) based on 10 copies/μg DNA. One test sample was ≧LLOQ—gag+IL-12 DNA—and one of two control samples was ≧LLOQ-IL-12 DNA—indicating an “unexpected positive”. After an extensive reexamination of the gag+HuIL-12 integration anaysis, it was recommended to increase the LLOQ from 10 to 100 copies/μg DNA to agree with industry standards for qPCR assay validation.

Both gag and HuIL-12 qPCR were performed on the high molecular weight DNA after four rounds of gel electrophoresis that separated the plasmid DNA. All the skin samples tested showed that the levels of both gag and HuIL-12 sequences-were below the LLOQ.

Clinical

Study of HIV CTL MEP+RC529-SE+/−GM-CSF (056) and Rollover Study (061)

The objectives of this study are to test safety, tolerability, and immunogenicity of HIV CTL MEP/RC529-SE±GM-CSF. The double-blind placebo-controlled study is being performed in HIV-negative healthy adults. To facilitate evaluation of cellular immune responses by tetramer analysis, individuals are screened for possession of at least one of three specified HLA alleles (A3, B7, or B8). Vaccine will be administered I.M. at 0, 4 and 12 weeks. CTL MEP adjuvant mixtures are made at the time of injection. The vaccine will be tested initially in two pilot groups (Part A), consisting of 10 actives and 2 placebos each for peptide/RC529-SE±GM-CSF. A safety evaluation was conducted at two weeks post dose two (day 42) before moving into additional subjects in Part B (96 total subjects). All 24 individuals in Part A were enrolled and had received 3 vaccinations at the time 29 individuals in Part B were enrolled (27 received 1 vaccination and 2 received 2 vaccinations). (See Table 9.) In addition to clinical safety evaluations, serum samples and peripheral blood mononuclear cells (PBMC) are taken for immunogenicity evaluation at multiple timepoints. Planned clinical assays include IFN-gamma ELISpot, intracellular cytokine staining, class I tetramer analyses, and antibody to GM-CSF.

TABLE 9

| | No. Subjects | | | |
|---|---|---|---|---|
| | (No. receiving | | | |
| | peptides + | | | |
| | specified | Total dose of | | |
| | adjuvants/No. | tetravalent | | |
| | receiving RC529-SE ± | peptides | RC529-SE | GM-CSF |
| Cohort | GM-CSF control) | (μg) | (μg) | (μg) |
| Part A | | | | |
| 1 | 10/2 | 1.000 | 50 | 0 |
| 2 | 10/2 | 1.000 | 50 | 250 |
| Part B | | | | |
| 3 | 30/6 | 1.000 | 50 | 0 |
| 4 | 30/6 | 1.000 | 50 | 250 |

Subjects from the 056 Study will be rolled over into the 061 Study and randomized to receive booster immunization with either gag plus HuIL-12 DNA or homologous peptide+/−GM-CSF at approximately months 8 and 11 (n=20/4 active/placebo per group). Timing to initiate the booster immunization phase of the rollover study is linked to completion of the HuIL-12 DNA dose escalation phase of the 060 Study (see below). HIV-specific T-cell responses will be assessed using IFN-gamma ELISpot assays, intracellular cytokine staining, and tetramer-binding assays.

Blinded safety data from 52 enrolled subjects was reviewed. All 52 subjects had received their first immunization, 27 of the 52 subjects (includes 2 subjects from part B) had received a second dose of study vaccine and 24 of the 52 subjects (all from part A) had received a third dose of study vaccine.

The majority of 12 pauses in enrollment/vaccination have been the result of preset criteria. In the first few days after each injection, mild to moderate pain and/or tenderness at the site where the injection was given have been reported in most individuals. Several individuals reported mild redness and/or swelling at the site of the injection. In a few cases after the first dose individuals reported severe pain and/or tenderness on the day of and/or day after vaccination improving over the next several days. In most participants, these side effects were gone with a day or two.

A low level interferon-gamma ELISPOT response was observed in a few subjects during a Phase I clinical trial of the 4-valent Th-CTL peptide with the RC529/GM-CSF adjuvant combination. No subjects demonstrated a response in a tetramer assay. Most subjects demonstrated an antibody response. Accordingly, the adjuvant combination may not be working as well as it did in pre-clinical animal studies.

All documents and other information sources cited herein are hereby incorporated in their entirety by reference. Also incorporated by reference is U.S. application Ser. No. 09/775,805 filed Feb. 5, 2001.

One skilled in the art will appreciate from a reading of this disclosure that various changes in form and detail can be made without departing from the true scope of the invention.

TABLE 1
Frequencies of HLA Class I Alleles That are Known to Serve
as HIV CTL Restriction Elements in Four Populations

| | Frequencies* | | | |
|---|---|---|---|---|
| HLA | African | USA | North American | |
| Alleles | Americans | Caucasians | Indians | Thais |
| A2 | 16.7 | 28.3 | 25.5 | 25.5 |
| A3 | 8.9 | 12.2 | 2.9 | 1.5 |
| A11 | 2.3 | 5.5 | 1.0 | 32.5 |
| A24 | 4.7 | 9.6 | 19.6 | 14.6 |
| A28 | 10.9 | 4.5 | 6.9 | 0.8 |
| A30 | 9.5 | 2.6 | 2.0 | 1.1 |
| A31 | 1.7 | 2.0 | 27.5 | 1.7 |
| A32 | 1.0 | 5.1 | 2.0 | 0.2 |
| A33 | 8.1 | 1.0 | 1.0 | 13.6 |
| B7 | 8.3 | 10.0 | 3.9 | 2.7 |
| B8 | 3.2 | 10.0 | 5.6 | 0.2 |
| B12 (44) | 6.2 | 10.4 | 3.9 | 5.4 |
| B13 | 0.9 | 3.0 | 1.0 | 9.3 |
| B14 | 3.0 | 4.1 | 2.9 | 0.4 |
| B17 | 10.9 | 4.9 | 1.0 | 8.1 |
| B18 | 3.3 | 4.9 | 1.0 | 2.5 |
| B27 | 1.6 | 4.1 | 2.9 | 6.0 |
| B35 | 7.7 | 8.5 | 18.6 | 2.5 |
| B37 | 0.9 | 2.2 | 0.0 | 1.4 |
| B52 | 1.1 | 1.2 | 2.9 | 3.1 |
| B53 | 12.8 | 0.8 | 0.0 | 0.0 |
| B57 | 4.2 | 3.9 | 1.0 | 5.2 |
| B60 | 1.3 | 4.5 | 2.9 | 8.3 |
| B62 | 1.4 | 5.5 | 4.9 | 5.0 |
| Cw3 | 9.6 | 12.6 | 22.4 | 15 |
| Cw4 | 21.0 | 9.8 | 15.4 | 6 |

*Frequencies for HLA-A and HLA-B alleles are taken from HLA 1991 (21). HLA-C for African Americans and USA Caucasians are taken from Histocompatibility Testing 1984 (19), HLA-C for North American Indians from Williams and McAuley, 1992 (22), and HLA-C for Thais from the Proceedings of the Second Asia and Oceania Histocompatibility Workshop Conference (23).

TABLE 2

Proportion of each of the four populations that would be predicted
to present peptides to the immune system

| | HLA Restriction | HIV | Epitope | |
|---|---|---|---|---|
| Population | Elements Chosen | Protein | Location | Epitope |
| a) African Americans | A2, A3, A11, B35 | nef | 73-82 | QVPLRPMTYK |
| | A28, B14 | gp41 | 583-592 | VERYLKDQQL |
| | A30, B8 | gp41 | 844-863 | RRIRQGLERALL |
| | B17, B37 | nef | 117-128 | TQGYFPDWQNYT |
| | Cw4 | gp120 | 576-383 | (S)FNCGGEFF |

(Proportion of African Americans expected to present these 5 epitopes is 92.3%)

| | | | | |
|---|---|---|---|---|
| b) USA Caucasians | A2, A3, A11, B35 | nef | 73-82 | QVPLRPMTYK |
| | A30, B8 | gp41 | 844-863 | RRIRQGLERALL |
| | B7 | gp120 | 302-312* | RPNNNTRKSI |
| | | nef | 126-138* | NYTPGPGVRYPLT |
| | B12 | p24 | 169-184 | IPMFSALSEGATPQDL |

(Proportion of USA Caucasians expected to present these 4 epitopes is 90.2%)

| | | | | |
|---|---|---|---|---|
| c) North American | A2, A3, A11, B35 | nef | 73-82 | QVPLRPMTYK |
| Indians | A24 | gp41 | 584-591* | YLKDQQL |
| | | nef | 120-144* | YFPDWQNYTPGPGIRYPLTFGWCYK |
| | A31 | gp41 | 770-780 | RLRDLLLIVTR |

(Proportion of North American Indians expected to present these 3
epitopes is 96.4%)

| | | | | |
|---|---|---|---|---|
| d) Thais | A2, A3, A11, B35 | nef | 73-82 | QVLRPMTYK |
| | A24 | gp41 | 584-591* | YLKDQQL |
| | | nef | 120-144* | YFPDWQNYTPGPGIRYPLTFCGWCYK |

(Proportion of Thais expected to present these 2 epitopes is 93.6%)

| | | | | |
|---|---|---|---|---|
| e) African Americans | A2, A3, A11, B35 | nef | 73-82 | QVPLRPMTYK |
| USA Caucasians | A28, B14 | gp41 | 583-592 | VERYLKDQQL |
| North American | | | | |
| Indians | A30, B8 | gp41 | 844-863 | RRIRQGLERALL |
| Thais | B17, B37 | nef | 117-128 | TQGYFPDWQNYT |
| | Cw4 | gp120 | 376-383 | (S)FNCGGEFF |
| | B7 | gp120 | 302-312* | RPNNNTRKSI |
| | | nef | 126-138* | NYTPGPGVRYPLT |
| | B12 | p24 | 169-184 | IPMFSALSEGATPQDL |
| | A31 | gp41 | 770-780 | RLRDLLLIVTR |
| | A24 | gp41 | 584-591* | YLKDQQL |
| | | nef | 120-144* | YFPDWQNYTPGPGIRYPLTFCGWCYK |

(Proportions of African Americans, USA Caucasians, North American Indians,
and Thais expected to present these 9 epitopes are 95.4%, 97.5%, 99.4%,
and 97.2%, respectively)

*The criteria upon which choices among peptides should be made are not yet known. It may be important to choose peptides that have been reported to be immunogenic in non-progressors to AIDS or that have been reported to induce immunodominant anti-HIV T-cell responses.

TABLE 3

Th-CTL Peptide Prototype Vaccine Immunogens for Testing in
Either Mice, Rhesus Macaque or Human

| Vaccine | | Species in which | | Restricting elements for |
|---|---|---|---|---|
| number | Name of Peptides | to be studied | Amino acid sequence | CTL epitope |
| 1. | Mouse HIV-1 Th-CTL | | Th-CTL | |
| | epitopes | | | |
| | A-Th/A-CTL | Mouse | HAGPTAPGQMREPRG- | H-2^{dd} |
| | | | KQIINMWQEVGKAMYA | |
| | B-Th/B-CTL | Mouse | KEKVYLAWVPAHKGIG- | H-2 K^{d} |
| | | | MYAPPIGGQI | |
| | C-Th/C-CTL | Mouse | QLLFIHFRIGCRHSR- | H-2^{d,p,a,q} |
| | | | DRVIEVVQGAYRAIR | (D^{d}) |
| | d-Th/D-CTL | Mouse | EQMHEDIISLWDQSL- | H-2 D^{d} |
| | | | RHHGPGRAFYTTKN | |
| 3. | Macaque SIV/HIV-1 Th- | | Th-CTL | |
| | CTL epitopes | | | |
| | Th1/CTL/SIV Gag | Macaque | ELYKYKVVKIEPLGVAPTKA- | Mamu-A*01 |
| | | | CTPYDINQM | |
| | Th2/CTL/SIV Po1 | Macaque | VSTVQCTHGIRPVVSTQLLL- | Mamu-A*01 |
| | | | STPPLVRL | |
| | Th3/CTL/HIV-1 Env | Macaque | STSIRGKVQKEYAFFYRLDI- | Mamu-A*01 |
| | | | YAPPISGQI | |
| 5. | Macaque SIV/HIV-1 Th- | | Th-CTL | |
| | CTL,pIle epitopes variants | | | |
| | Th1/CTL/SIV Gag | Macaque | ELYKYKVVKIEPLGVAPTKA- | Mamu-A*01 |
| | | | CTPYDINQM | |
| | Th2/CTL/SIV Gag/pIle/1-Y | Macaque | VSTVQCTHGIRPVVSTQLLL- | Mamu-A*01 |
| | | | CTPYDYNQML | |
| | Th3/CTL/SIV Gag/pIle/1-A | Macaque | STSIRGKVQKEYAFFYKLDI- | Mamu-A*01 |
| | | | CTPYDANQML | |
| | Th4/CTL/SIV Gag/pIle/1-D | Macaque | EYAFFYKLDIIPIDNDTTSY- | Mamu-A*01 |
| | | | CTPYDDNQML | |
| | Th5/CTL/SIV Gag/pIle/1-K | Macaque | REQFGNNKTIIFKQSSGGDPE- | Mamu-A*01 |
| | | | CTPYDKNQML | |
| 6. | Human HIV-1 Th-CTL | | Th-CTL | |
| | overlapping epitopes | | | |
| | A-Th/A-CTL | Human | KQIINHWQEVGKAMYA- | HLA, B57,B58 |
| | | | KAFSPEVIPHF | |
| | B-Th/B-CTL | Human | YKRWIILGLNKIVRHYS- | HLA,B35,B8,B27, |
| | | | HPPIPVGEIYKRWI- | A33,Bw62,B52 |
| | | | ILGLNKIVRMYSPTSI | |
| | C-Th/C-CTL | Human | DRVIEVVQGAYRAIR- | HLA,A1,B7,B8, |
| | | | VGFPVRPQVPLRPMTYK | B35,A11,A2,A3, |
| | | | | A31 |
| | D-Th/D-CTL | Human | ASLWNWFMIHWLWY- | HLA,B7,B57,A1, |
| | | | WVYHTQGFFPDWQHYTP | B8,B18,B35 |
| 8. | Human HIV-1 Th- | | Th-CTL | |
| | dominant/subdominant | | | |
| | CTL epitopes | | | |
| | A-Th/E-CTL | Human | KQIINMWQEVGKAMYA- | HLA A2 |
| | | | SLYNTVATL | |
| | B-Th/F-CTL | Human | YKRWIILGLNKIVRHYS- | HLA A3 |
| | | | KIRLRPGGK | |
| | C-Th/G-CTL | Human | DRVIEVVQGAYRAIR- | HLA B27 |
| | | | KRWIILGLNK | |
| | D-Th/H-CTL | Human | ASLWNWFNITNWLWY- | HLA B8 |
| | | | GGKKKYKL | |
| | E-Th-I-CTL | | MREPRGSKIAGTTST- | HLA B14 |
| | | | ERYLKDQQL | |
| 10. | Human HIV-1 Th-CTL | | Th-CTL | |
| | p17 epitope (A2 Variants) | | | |
| | B-Th/E-CTL | Human | YKRWIILGLNKIVRMYS- | HLA A2 |
| | | | SLYNTVATL | |
| | C-Th/J-CTL | Human | DRVIEVVQGAYRAIR- | HLA A2 |
| | | | SLFNTVATL | |
| | A-Th/K-CTL | Human | QIINMWQEVGKAMYA- | HLA A2 |
| | | | SLYNAVATL | |
| | D-Th/L-CTL | Human | ASLWNWFNITNWLWY- | HLA A2 |
| | | | SLYHTVAVL | |
| | E-Th/M-CTL | Human | MREPRGSKIAGTTST- | HLA A2 |
| | | | SLFNLLAVL | |

| | | | |
|---|---|---|---|
| 11. | Human HIV-1 Th-CTL | Th-CTL | |
| | ovelapping epitopes | | |
| | A*-Th/J-CTL | KQIINMWQVVGKAMYA- | A2,A202,A5,B7, |
| | | GQMVHQAISPRTLNAWVKVV | B14,B57,B5701, |
| | | | B5801, B02, Cw3 |
| | A*-Th/K-CTL | KQIINMWQVVGKAMYA- | A2,A25,A26,B7, |
| | | ATPQDLNTMLNTVGGHQAAMQ | B12,B14,B1402, |
| | | MLKETINEEAAEW | B27,B39,B52,B53, |
| | | | B57,B58,B8101, |
| | | | Cw8,Cw0102 |
| | A*-Th/L-CTL | KQIINMWQVVGKQAMYA- | A2,A202,A5,A24, |
| | | GPKEPFRDYVDRFYKTLRAEQ | A2402,A25,A26, |
| | | ASQEVKNWMT | A33,B7,B8,B12,B14, |
| | | | B35,B39,B44,B52, |
| | | | B53Bw62,B27,B2705, |
| | | | B57,B5701,70,B71, |
| | | | Bw62,Cw3,Cw8,Cw0401 |
| | A*-Th/M-CTL | KQIINMWQVVGKAMYA- | A1,A2,A3,A01,A03, |
| | | KIRLRPGGKKKYKLKHIVWGSE | A11,A23,A24,A0201, |
| | | ELRSLYNTVATLYCVHQRI | A2402,B8,B27,B42, |
| | | | B62,Bw62,Cw4 |

TABLE 4
Linear Array of Th-CTL Epitopes To Be Expressed in Modified Vaccinia Ankara

```
MVA-1) HIV-1 mouse Tb-CTL epitopes in

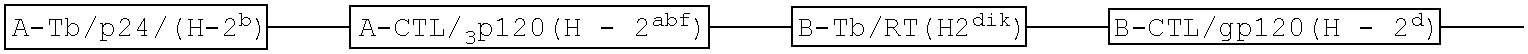

  HAGPIAPGQMREPRG--KQIINMWQEVGKAMYA----KEKVYLAWVPAMKGIG----MYAPPIGGQI-

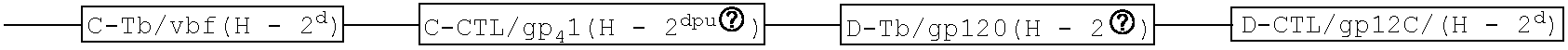

--QLLFIHRIGCRHSR---DRVIEVVQGAYRAIR----EQMMEDIISLWDQSL---RIHIGPGRAFYTTKN
MVA-2) p55/gag + the same HIV-1 mouse Th-CTL epitopes in MVA-1
MVA-3) HIV-1/SIV Th-CTL epitopes in

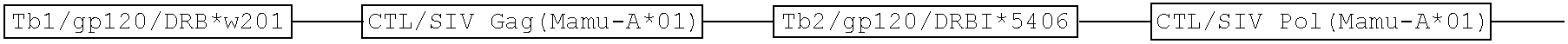

ELYKYKVVKIEPLGVAPTKA-------CTPYDINQM--------VSTQCTHGIRPVVSTQLLL-----STPPLVRL-

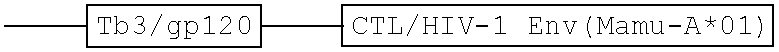

    --STSIRGKVQKEYAFFYKLDI--------YAPPISGQI
MVA-4) p55/gag + the same HIV-1/SIV Th-CTL epitopes in MVA-3
MVA-5) SIV Th-CTL p11c epitope variants in

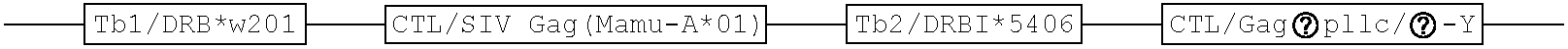

ELYRYKVVKIEPLGVAPTKA----CTPYDINQML-------VSTQCTHGIRPVVSTQLLL----CTPDYNQML-

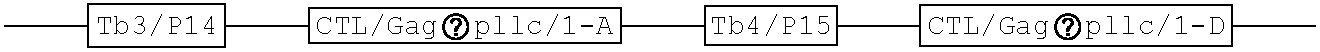

-STSIRGKVQKEYAFFYLQI---CTPYDANQML------EYAFFYKLDIIPIDNDTTSY------CTPYDINQML-

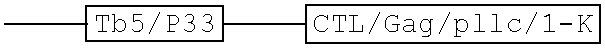

      -REQFGNNKTIIFKQSSGGDPE----CTPYDKNQML
MVA-6) HIV-1 human Th-CTL overlapping epitopes in

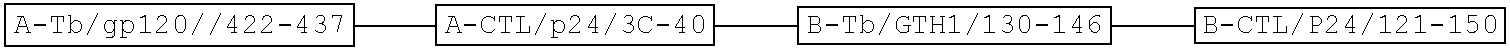

KQIINMWQEVGKAMYA----KAFSPEVIPMF----YKRWIILGLNKIVRMYS----NPPIPVGEIYKRWIILGLNKIVRMYSPTSI-

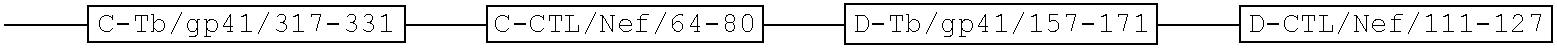

--DRVIEVVCGAYRAIR---VGFPVRPQVPLRPMTYK---ALSWNWFNITNWLWY----WVYHTQGFFPDWQNYTP
Restricting elements for CTL epitopes:
A-CTL epitopesHLA B57/B58. B-CTL epitopesHLA B35/B8/B27/A33/Bw62/B52;
C-CTL epitopesHLA A1/B7/B8/B35/A11/A2/A3/A31); D-CTL epitopesHLA B7/B57/A1/B8/B18/B35.
MVA-7) p55 gag + the same HIV-1 human Th-CTL overlapping epitopes in MVA-6
MVA-8) HIV-1 Th-domain/subdominant CTL epitopes in

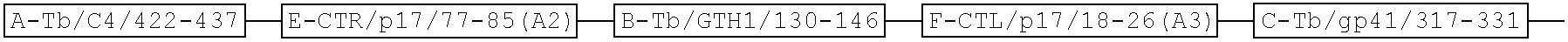

KQIINMWQEVGKAMYA-----SLYNTVATL-----YKRWIILGLNKIVRMYS----KIRLRPGGK------DRVIEVVQGAYRAIR-

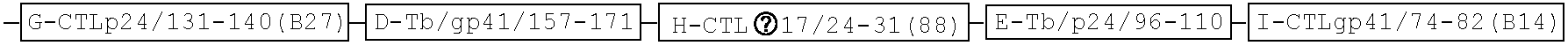

  --KRWIILGLNK-----ASLWNWFNITNLWLY-----GGKKKYKL------MREPRGSKIAGTTST----ERYLKDQQL-
MVA-9) p55/gag + the same HIV-1 Th-domain/ssubdominant CTL epitopes in MVA-8
MVA-10) HIV-1 Th-CTL A2 p17 epitope (A2 Variants) in

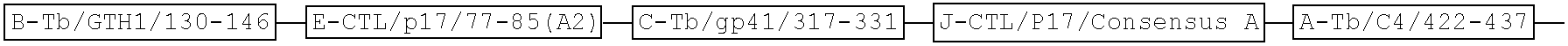

YKRWIILGLNKIVRMYS----SLYNTVATL------DRVIEVVQGAYRAIR----SLFNTVATL-------KQIINMWQEVGKAMYA-

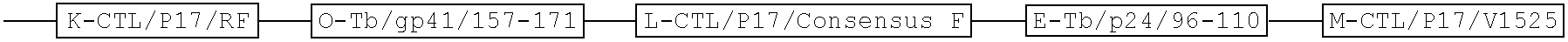

--SLYNAVATL----ASLWNWFNITNWLWY-------SLYNTVAVL--------MREPRGSKIAGTTST-----SLFNLLAVL
```

TABLE 5

HIV Polyvalent C4-V3 Peptides Studied in
Guinea Pigs, Primates Or In Humans

| | |
|---|---|
| Peptide | gp120 C4 Region gp120 V3 Region |

| | |
|---|---|
| C4-V3MN | KQIINMWQEVGKAMYATRPNYNKRKRIHIGPGRAFYTTK |
| C4-V3RF | KQIINMWQEVGKAMYATRPNNNTRKSITKGPGRVIYATG |
| C4-V3EV91 | KQIINMWQEVGKAMYATRPGNNTRKSIPIGPGRAFIATS |
| C4-V3CanOA | KQIINMWQEVGKAMYATRPHNNTRKSIHMGPGKAFYTTG |
| C4E9G-V3RF | KQIINMWQGVGKAMYATRPNNNTRKSITKGPGRVIYATG |
| C4E9V-V3RF | KQIINMWQVVGKAMYATRPNNNTRKSITKGPGRVIYATG |
| C4K12E-V3RF | KQIINMWQEVGEAMYATRPNNNTRKSITKGPGRVIYATG |

Sequences from the Los Alamos Database.

TABLE 6
Th-CTL Peptide Prototype Vaccine Immunogens derived from HIV-1 gag

| Vaccine | | | Restricting elements for |
|---|---|---|---|
| number | Name of Peptides | Amino acid sequence | CTL epitope |
| | Human HIV-1 Th-CLT | Th-CTL | |
| | overlapping epitopes | | |
| 6 | A-Th/A-CTL | KQIINMWQEVGKAMYA-KAFSPEVIPMF | B57,B58 |
| 6 | B-Th/B-CTL | YKHWIILGLNKIVRMYS- | B35,B8,B27,A33,Bw62,B52 |
| | | NPPIPVGEIYKRWIILGLNKIVRMYSPTSI | |
| 11 | A*-Th/J-CTL | KQIINMWQVVGKAMYA- | A2,A202,A5,B7,B14,B57,B5701, |
| | | GQMBHQAISPRTLNAWVKVV | B5801,B02,Cw3 |
| 11 | A*-Th/K-CTL | KQIINMWQVVGKAMYA- | A2,A25,A26,B7,B12,B14,B1402, |
| | | ATPQDLNTMLNTVGGHQAAMQMLKETINEEAAEW | B27,B19,B52,B53,B57,B58,B8101,Cw8, |
| | | | Cw0102 |
| 11 | A*-Th/L-CTL | KQIINMWQVVGKAMYA- | A2,A202,A5,A24,A2402,A25,A26, |
| | | GPKEPFRDYVDRFYKTLRAEQASQEVKNWMT | A33,B7,B8,B12,B14,B35,B39,B44,B52, |
| | | | B53Bw62,B27,B2705,B57,B5701,B70, |
| | | | B71,Bw62,Cw3,Cw8,Cw0401 |
| 11 | A*-Th/M-CTL | KQIINMWQVVGKAMYA- | A1,A2,A3,A3.1,A03,A11,A23,A24,A0201, |
| | | KIRLRPGGKKKYKLKHIVWGSEELRSLYNTVATL | A2402,B8,B27,B42,B62,Bw62,Cw4 |
| | | YCVHQRI | |

A*-Th = C4E9V
Summary of restracting elements for CTL, epitopes in vaccines A, B, J, K, K, L and M
A: A1, A2 (02), (01), A3, A3.1, A5, A11, A23, A24 (02), A25, A26 and A33.
B: B7, B8, B12, B14 (02), B27 (05), B35, B39, B42, B44, B52, B53, B57 (01), B58 (01), B62 (wb2), B70 and B71.
C: Cw3, Cw4, Cw0401 and Cw8.

TABLE 7

| | | | | |
|---|---|---|---|---|
| | | Restricting elements for | African | USA |
| Name of Peptides | Amino acid sequence of CTL | CTL epitope | American | Caucasians |

| | | | | | |
|---|---|---|---|---|---|
| A*-Th/M1-CTL | Gag 18-36 | KIRLRPGGKKKYKLKHIVW | A3, Cw4, B8, A3,Bw62, | | |
| | (P17 18-36) | | B62, B42, A30, A3.1 | | |
| | | | A*0301,B27 B*2705 | 30.3 | 35.06 |
| A*-Th//M2-CTL | Gag 70-92 | TGSEELRSLYNTVATLYCVHQRI | A11, A*1101, A201, B62, | | |
| | (P17 70-92) | | B*0201, A2.1 A2, | | |
| | | | A*0201,A*0205, A*02, | | |
| | | | A30, A*3002, B8, B*0801, | | |
| | | | A1 | 38.8 | 67.1 |
| B-Th/L2-CTL | Gag 291-319 | EPFRDYVDRFYKTLRAEQASQEVKNW | B44, B*4402, Cw8, B14, | | |
| | (P24 159-187) | MTE | B*1402,B70B*1510,A26, | | |
| | | | A*0207, A24, A*2402, | | |
| | | | B71, B18, B*1801, | | |
| | | | B*44031, B53 | 32.8 | 33.7 |
| A*-Th/Q-CTL | Nef 180-198 | VLVWRFDSRLAFHHMAREL | A1, A3, A2, A*0202, | | |
| | | | A*0201, B35, C4, B52, | | |
| | | | B51, A24, B*1503, B35, | | |
| | | | A25, B8, A*0201, B7, H-2d | 59.3 | 92.452 |
| B-Th/R-CTL | Pol 312-343 | SPAIFQSSMTKILEPFRKQNPDIVIY | A3,B*0301, A33, A3.1, | | |
| | | QYMDDL | A11, A*1101, A*6801, B7, | | |
| | | | B35, B*3501, B*5101, | | |
| | | | A*0201, A2, A*0202, B51, | | |
| | | | B*2002 | 54.6 | 68.1 |
| B-Th/T-CTL | Env 33-61 | KLWVTVYYGVPVWKEATTTLFCASDA | B35, B*3501, B55, | | |
| | | KAY | B*5501, Cw7, B*0301, | | |
| | | | A3.1 A3 A11,A03. | | |
| | | | A*0201, A11, A*6801, | | |
| | | | A2.1, A2, B44, 8*4402, | | |
| | | | B38,A24,A*2402 | 47 | 76.44 |

B-Th: YKRWIILGLNKIVRMYS

TABLE 8

| | Location of | | |
|---|---|---|---|
| | CTL ″hot | | |
| Name of Peptide | spot″¹ | a.a. Sequence | Restricting elements for CTL epitope |
| A*-Th/L2-CTL | HIV gag (p24 | KQIINMWQVVGKAMYA- | A*0201, A*0207, A*2402, A*26, B*1402, |
| | 159-187) | EPFRDYVDRFYKTLRAEQASQEVKNWMTE | B*1510, B*1801, B*4402, B*44031, |
| | | | B*5301, B*5701, B*70, B*71, Cw4, Cw8 |
| A*-Th/M1-CTL | HIV gag (p17 | KQIINMWQVVGKAMYA- | A*0201, A*0301, A*23, A*2402, A*30, |
| | 18-36) | KIRLRPGGKKKYKLKHIVW | B*0301, B*7, B*1801,B*2705, B*42, |
| | | | B*62, Cw4 |
| A*-Th/M2-CTL | HIV gag (p17 | KQIINMWQVVGKAMYA- | A1, A*0201, A*0202, A*0205, A*0214, |
| | 70-92) | TGSEELRSLYNTVATLYCVHQRI | A*1101, A*3002, B*0201, B*0301, B*62 |
| A*-Th/R-CTL | HIV pol | KQIINMWQVVGKAMYA- | A*0202, A*03 supertype, A*1101, A43002, |
| | (312-343) | SPAIFQSSMTKILEPFRKQNPDIVIYQYMDDL | A*33, A*6801, B*0301, B*07, B*3501, B*51 |

¹CTL hot spot locations are based on the Los Alamos National Laboratory, ″HIV Molecular Immunology 2002: Maps of CTL Epitope Locations Plotted by Protein″, December 12, 2003.
*Designates a universal T-helper epitpe with an E-V substitution at position 9.

What is claimed is:

1. A peptide selected from the group consisting of A*Th/M1-CTL, A*Th/M2-CTL, B-Th/L2-CTL, A*Th/Q-CTL, B-Th/R-CTL, B-Th/T-CTL, A*Th/L2-CTL, and A*Th/R-CTL.

2. The peptide according to claim 1 wherein said peptide is A*Th/M1-CTL.

3. The peptide according to claim 1 wherein said peptide is A*Th/M2-CTL.

4. The peptide according to claim 1 wherein said peptide is B-Th/L2-CTL.

5. The peptide according to claim 1 wherein said peptide is A*Th/Q-CTL.

6. The peptide according to claim 1 wherein said peptide is B-Th/R-CTL.

7. The peptide according to claim 1 wherein said peptide is B-Th/T-CTL.

8. The peptide according to claim 1 wherein said peptide is A*Th/L2-CTL.

9. The peptide according to claim 1 wherein said peptide is A*Th/R-CTL.

10. A composition comprising at least one peptide selected from the group consisting A*Th/M1-CTL, A*Th/M2-CTL, B-Th/L2-CTL, A*Th/Q-CTL, B-Th/R-CTL, B-Th/T-CTL, A*Th/L2-CTL, and A*Th/R-CTL, and a carrier.

11. The composition according to claim 10, wherein said composition comprises at least the peptides A*Th/M1-CTL and A*Th/M2-CTL.

12. The composition according to claim 11 wherein said composition further comprises at least one peptide selected from the group consisting of B-Th/L2-CTL and B-Th/R-CTL.

13. The composition according to claim 11, wherein said composition further comprises at last one peptide selected from the group consisting of A*Th/L2-CTL and A*Th/R-CTL.

14. The composition according to claim 10 wherein said composition further comprises an adjuvant.

15. A nucleic acid encoding a peptide selected from the group consisting of A*Th/M1-CTL, A*Th/M2-CTL, B-Th/L2-CTL, A-Th/Q-CTL, B-Th/R-CTL, B-Th/T-CTL, A*Th/L2-CTL, and A*Th/R-CTL.

16. A construct comprising the nucleic acid according to claim 15 and a vector.

17. The construct according to claim 16 wherein said vector is a viral vector.

18. A composition comprising the nucleic acid according to claim 15 and a carrier.

19. A method of inducing an immune response in a mammal comprising administering to said mammal an amount of said peptide according to claim 1 sufficient to effect said induction.

20. A method of inducing an immune response in a mammal comprising administering to said mammal an amount of said nucleic acid according to claim 15 sufficient to effect said induction.
